(19) United States
(12) Patent Application Publication
(10) Pub. No.: US 2017/0304223 A1
(43) Pub. Date: Oct. 26, 2017

(54) METHOD OF TREATING PROSTATE CANCER USING A PKC INHIBITOR

(71) Applicants: Mildred Acevedo-Duncan, Plant City, FL (US); Andre Apostolatos, Lutz, FL (US)

(72) Inventors: Mildred Acevedo-Duncan, Plant City, FL (US); Andre Apostolatos, Lutz, FL (US)

(21) Appl. No.: 15/495,527

(22) Filed: Apr. 24, 2017

Related U.S. Application Data
(60) Provisional application No. 62/326,366, filed on Apr. 22, 2016.

(51) Int. Cl. A61K 31/122

(52) U.S. Cl. CPC A61K 31/122

(57) ABSTRACT

The invention relates to compositions comprising a PKC inhibitor and their use in a method of treating or preventing the development of prostate cancer.

### CROSS-REFERENCE TO RELATED APPLICATIONS

This application claims priority to U.S. Provisional Application No. 62/326,366 filed on Apr. 22, 2016, which is incorporated fully herein by reference.

### TECHNICAL FIELD

This invention relates to treating cancer with inhibitors of atypical protein kinase C. Specifically, the invention describes methods of treating prostate cancer using a protein kinase C (PKC) inhibitor such as 2-acetyl-1,3-cyclopentanedione (ACPD) or ICA-1.

### BACKGROUND

The American Cancer Society estimates 220,800 new cases of prostate cancer (PC) will arise in 2016. Prostate cancer is the second leading cause of death among American men. An approximated 1 out of 7 men will be diagnosed with the disease during their lifetime and 1 out of 38 will die of it. Although there have been advancements in the treatment of PC which have improved overall survival, patients frequently develop resistance to treatment and incur unwanted side effects that negatively impact the patient's lifestyle. As such, alternative therapeutic agents for the treatment of prostate cancer are needed.

### SUMMARY OF INVENTION

The invention discloses a method of treating or preventing the development of prostate cancer in a subject, comprising administering to the subject a composition comprising a protein kinase C (PKC) inhibitor. The subject may be a mammal, such as a human. The PKC inhibitor may inhibit at least one atypical PKC (aPKC). The PKC inhibitor may inhibit PKC-iota. For example, the PKC inhibitor may be ICA-1 or a salt thereof. The PKC inhibitor may be a pan-aPKC inhibitor. For example, the pan-aPKC inhibitor may be ACPD or a salt thereof. The PKC inhibitor may be administered in combination with at least one other anti-cancer therapy.

### BRIEF DESCRIPTION OF THE DRAWINGS

FIGS. 1A-C show PKC-α and PKC-τ expression in benign prostate hyperplesia (BPH) and malignant prostate cancer (PC) tissues.

FIGS. 2A-F show IHC staining of PKC-α and PKC-τ in benign, high grade PIN and malignant prostate tissue.

FIGS. 3A-B show the inhibition of PKC-τ 72 hours after treatment with either ICA-1 or ACPD.

FIGS. 4A-B show the dose curves generated to determine the concentration of drugs needed for an effective response in malignant cancer cells while having no significant effect on control cells.

FIGS. 5A-C show cell population of RWPE control cells and DU-145 cancer cells following treatment with ICA-1 or ACPD.

FIG. 6 shows levels of apoptotic markers in RWPE control cells and DU-145 cancer cells following treatment with ICA-1 or ACPD.

FIG. 7 shows NF-kB levels in the cytosol and nucleus of DU-145 and PC-3 cell lines following treatment with ICA-1 or ACPD.

### DETAILED DESCRIPTION

Protein kinase C (PKC) is a family of isozymes that transduce signals and control other proteins through phosphorylation. PKC isozymes can be classified into three groups. Group I includes Ca²⁺ dependent isozymes: cPKC-alpha, cPKC-betaI cPKC-betaII and cPKC-gamma. Isozymes in group II, nPKC-epsilon, nPKC-delta, nPKC-eta and nPKC-theta are Ca²⁺-independent. Group III includes the atypical PKCs (PKC-τ, PKC-ζ, PKC-ζll, PKC-μ, and PKC-ν) which are insensitive to both diacylglycerol and calcium and neither bind to nor are activated by tumor promoting phorbol esters. PKC is the major receptor for tumor promoting phorbol esters, but the extent of PKC involvement in cellular malignancy and prostate cancer is not clearly defined. The present disclosure describes a method of treating prostate cancer in a subject, comprising administering to the subject a composition comprising a protein kinase C (PKC) inhibitor.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “administration” or “administering” is used throughout the specification to describe the process by which the disclosed PKC inhibitor compositions may be delivered to a subject. Administration will often depend upon the amount of composition administered, the number of doses, and duration of treatment. Multiple doses of the composition may be administered. The frequency and duration of administration of the composition can vary, depending on any of a variety of factors, including patient response, etc. The PKC inhibitor compositions may be administered to the subject by any suitable route that allows the PKC inhibitor to contact tumor cells. The compositions may be administered orally, parentally, (including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, and epidural injection) by infusion, by electroporation, or co-administered as a component of any medical device or object to be inserted (temporarily or permanently) into a subject.

The PKC inhibitor composition may be administered independently or in combination with other anti-cancer therapies, including surgery, radiation therapy, chemotherapy, DNA therapy, adjuvant therapy, gene therapy, and other therapeutic agents for the treatment of prostate cancer.

The term “atypical protein kinase” or “aPKC” as used interchangeably herein refers to members of the PKC family that are insensitive to diacylglycerol and calcium. Atypical PKCs include PKC-τ, PKC-ζ, PKC-ζll, PKC-μ, and PKC-ν.

The term “cancer”, “neoplasia”, “tumor”, “cancerous”, and malignant” as used herein, refer to the physiological condition in mammals that is typically characterized by unregulated cell growth or the presence of tumors. Examples of cancer benefited by the present invention include, but are not limited to, ovarian cancer, breast cancer, prostate cancer and glioblastoma.

The term “carrier” as used herein refers to a diluent, adjuvant, excipient, or vehicle with which the PKC inhibitor is administered. Such pharmaceutical carriers can be sterile liquids, such as water and oils, including those of petroleum oil such as mineral oil; vegetable oil such as peanut oil, soybean oil, and sesame oil; animal oil; or oil of synthetic origin. Suitable carriers also include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

“Derivative” refers to a compound or portion of a compound that is derived from or is theoretically derivable from a parent compound.

The term “pan-aPKC inhibitor” as used herein refers to an agent that inhibits the activity or reduces/inhibits the expression of at least two atypical PKCs such as PKC-τ and PKC-ζ. Examples of such agents include, but are not limited to ACPD, pachastrissamine and its stereoisomers, and derivatives thereof.

The term “PKC inhibitor” as used herein refers to an agent that inhibits the activity or reduces/inhibits the expression of one or more isoforms of protein kinase C (PKC). The PKC inhibitor may be an atypical protein kinase C inhibitor (aPKC inhibitor). The inhibitor may be a polypeptide that binds to the one or more PKC isoforms and inhibits its activity. The inhibitor may be a polypeptide that is involved with the interaction of the one or more PKC isoforms with other signaling molecules. The inhibitor may be a polypeptide that mediates the binding other signaling molecules to the one or more PKC isoforms. The inhibitor may be a small molecule inhibitor.

A “pharmaceutically acceptable excipient,” “pharmaceutically acceptable diluent,” “pharmaceutically acceptable carrier,” or “pharmaceutically acceptable adjuvant” as used herein means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use, such as those promulgated by the United States Food and Drug Administration.

The disclosed PKC inhibitor compositions may comprise a pharmaceutically acceptable salt of a PKC inhibitor. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N+(C1-4alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, the PKC inhibitors disclosed herein are also meant to include all isomeric (e.g., enantiomeric, diastereomeric, and geometric (or conformational)) forms of the inhibitor; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present inhibitors are within the scope of the disclosure. Unless otherwise stated, all tautomeric forms of the inhibitors of the disclosure are within the scope of the disclosure. Additionally, unless otherwise stated, inhibitors depicted herein are also meant to include compounds that differ only in the presence of one or more isotopically enriched atoms. For example, the present inhibitors including the replacement of hydrogen by deuterium or tritium, or the replacement of a carbon by a 13C- or 14C-enriched carbon are within the scope of this disclosure.

The terms “PKC-τ inhibitor”, “PKC-iota inhibitor”, or “PKC iota inhibitor” as used interchangeably herein refers to an agent that inhibits PKC-τ activity or reduces or inhibits expression of PKC-τ. The agent may be specific to PKC-τ. For example, the PKC-τ inhibitor may be ICA-1 or a derivative thereof. The PKC-τ inhibitor may be a pan-aPKC inhibitor that is effective against multiple aPKC isoforms including PKC-τ. For example, PKC-τ inhibitor may be ACPD. The inhibitor may be a polypeptide that binds to PKC-τ and inhibits its activity. The inhibitor may be a polypeptide that is involved with the interaction of PKC-τ with other signaling molecules. The inhibitor may be a polypeptide that mediates the binding other signaling molecules to PKC-τ. The inhibitor may be a small molecule inhibitor, such as ICA-1 or a salt or derivative thereof.

The terms “PKC-ζ inhibitor”, “PKC-zeta inhibitor”, or “PKC zeta inhibitor” as used interchangeably herein refers to an agent that inhibits PKC-ζ activity or reduces or inhibits expression of PKC-ζ. The agent may be specific to PKC-ζ or alternatively may be a pan-aPKC inhibitor that is effective against multiple aPKC isoforms including PKC-ζ. For example, the PKC-ζ inhibitor may be ACPD. The inhibitor may be a polypeptide that binds to PKC-ζ and inhibits its activity. The inhibitor may be a polypeptide that is involved with the interaction of PKC-ζ with other signaling molecules. The inhibitor may be a polypeptide that mediates the binding other signaling molecules to PKC-ζ. The inhibitor may be a small molecule inhibitor. Examples of a PKC-ζ inhibitor include, but are not limited to, ZIP, ACPD, speciosterosulfates, sterolsulfates, or salts or derivatives thereof.

The term “precancerous prostate state” as used herein refers to any prostate condition which has the potential to develop into prostate cancer. Examples of precancerous prostate states include prostatic intraepithelial neoplasia (PIN), proliferative inflammatory atrophy (PIA), and atypical small acinar proliferation (ASAP).

The term “sample” as used herein refers to any physical sample that includes a cell or a cell extract from a cell, a tissue, or an organ including a biopsy sample. The sample can be from a biological source such as a subject or animal, or a portion thereof, or can be from a cell culture. Samples from a biological source can be from a normal or an abnormal organism, such as an organism known to be suffering from a condition or a disease state such as a neoplasm, or any portion thereof. Samples can also be from any fluid, tissue or organ including normal and abnormal (diseased or neoplastic) fluid, tissue or organ. Samples from a subject or animal can be used in the present invention as obtained by the subject or animal and processed or cultured such that cells from the sample can be sustained in vitro as a primary or continuous cell culture or cell line. A “tumor sample” is a sample that includes at least one cell derived from at least one tumor.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). The subject may be a human or a non-human. The subject or patient may be undergoing other forms of treatment.

The term “treatment”, “treat”, “treating” or any grammatical variation thereof as used herein, includes but is not limited to, ameliorating or alleviating a symptom of a disease or condition, reducing, preventing, suppressing, inhibiting, lessening, or affecting the progression and/or severity of an undesired physiological change or a diseased condition. For example, treatment may include any one or more of inhibiting neoplastic transformation of cells, slowing the growth and/or proliferation of cancer cells, reducing tumor size, reducing the number of cancer cells, inducing apoptosis in cancer cells, decreasing the level of one or more atypical protein kinases in the cancer cells, decreasing the level of PKC-τ or PKC-ζ in cancer cells, inhibiting or slowing the invasion of cancer cells into surrounding or neighboring tissues, inhibiting or slowing the metastatic spread of cancer cells into distant parts of the body, enhancing the therapeutic effect of chemotherapy medications, and prolonging cancer patient survival.

A “therapeutically effective amount,” or “effective dosage” or “effective amount” as used interchangeably herein unless otherwise defined, means a dosage of a drug effective for periods of time necessary, to achieve the desired therapeutic result. Compositions of the present invention may be used to effect a favorable change in the condition whether that change is an improvement or a complete elimination of symptoms due to neoplasia/cancer. For example, an effective amount may cause a 5%, 10%, 20%, 30%, 40%, 50%, 60%, 70%, 80%, 90%, 95%, 99% or 100% reduction in the rate of growth and/or proliferation of cancer cells. As another example, the effective amount may cause a 5%, 10%, 20%, 30%, 40%, 50%, 60%, 70%, 80%, 90%, 95%, 99% or 100% reduction in tumor size.

In accordance with the present invention, a suitable single dose size is a dose that is capable of preventing or alleviating a symptom in a subject when administered one or more times over a suitable time period. An effective dosage may be determined by a person skilled in the art and may vary according to factors such as the disease state, age, sex, and weight of the individual, and the ability of the drug to elicit a desired response in the individual. Therapeutically effective amounts for the disclosed PKC inhibitor compositions can be readily determined by those of ordinary skill in the art.

A therapeutically effective amount may be administered in one or more administrations (e.g., the PKC inhibitor composition may be given as a preventative treatment or therapeutically at any stage of disease progression, before or after symptoms, and the like), applications or dosages and is not intended to be limited to a particular formulation, combination or administration route. It is within the scope of the present disclosure that the disclosed PKC inhibitor compositions may be administered at various times during the course of treatment of the subject. The times of administration and dosages used will depend on several factors, such as the disease state, age, sex, and weight of the individual, and the ability of the composition to elicit a desired response in the individual. Administration may be adjusted according to individual need and professional judgment of a person administrating or supervising the administration of the compounds used in the present invention.

### 2. PKC Inhibitor Compositions

The disclosure provides compositions comprising a protein kinase C (PKC) inhibitor. The PKC inhibitor may be any agent which inhibits the activity or expression of one or more isoforms of PKC. The inhibitor may be a polypeptide that binds to the one or more PKC isoforms and inhibits its activity. The inhibitor may be a polypeptide that is involved with the interaction of the one or more PKC isoforms with other signaling molecules. For example, the polypeptide may inhibit the interaction of one or more PKC isoforms with other signaling molecules. Alternatively, the polypeptide may stimulate the interaction of one or more PKC isoforms with other signaling molecules. The inhibitor may be a polypeptide that mediates the binding of other signaling molecules to the one or more PKC isoforms. For example, the polypeptide may inhibit the binding of other signaling molecules to the one or more PKC isoforms. Alternatively, the polypeptide may stimulate the binding of other signaling molecules to the one or more PKC isoforms. The inhibitor may be a small molecule inhibitor.

The PKC inhibitor composition may comprise an atypical PKC (aPKC) inhibitor. The aPKC inhibitor may inhibit any one or more atypical protein kinases. The aPKC inhibitor may inhibit any one or more of PKC-τ, PKC-ζ, PKC-ζll, PKC-μ, and PKC-ν. For example, the aPKC inhibitor may be a PKC-τ inhibitor. For example, PKC-τ inhibitor may be ICA-1 or a salt or derivative thereof. The aPKC inhibitor may be a PKC-ζ inhibitor. Examples of PKC-ζ inhibitors include ZIP, ACPD, speciosterolsulfates, sterolsulfates, and derivatives thereof.

The aPKC inhibitor may be specific to a single atypical PKC. The aPKC inhibitor may be a pan-aPKC inhibitor that is effective against multiple aPKC isoforms. The pan-aPKC inhibitor may be any agent that reduces or inhibits activity or expression of the multiple aPKC isoforms. The pan-aPKC inhibitor may inhibit any combination of aPKCs. The pan-aPKC inhibitor may inhibit any one or more of PKC-τ, PKC-ζ, PKC-ζll, PKC-μ, and PKC-ν. For example, the pan-aPKC inhibitor may inhibit PKC-τ and PKC-ζ. For example, the pan-aPKC inhibitor may be ACPD or a salt or derivative thereof. The pan-aPKC inhibitor may be pachastrissamine or a derivative thereof.

The PKC inhibitor composition may further comprise a pharmaceutically acceptable carrier or excipient. Such pharmaceutical carriers may be sterile liquids, such as water and oils. For example, the carrier may be a petroleum oil such as mineral oil; vegetable oil such as peanut oil, soybean oil, or sesame oil; animal oil; or oil of synthetic origin. Suitable carriers also include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The PKC inhibitor composition may contain minor amounts of wetting or emulsifying agents. The PKC inhibitor composition may contain pH buffering agents.

The PKC inhibitor composition may be in a variety of forms. For example, the PKC inhibitor composition may be in solid form, semi-solid form, or a liquid dosage forms. The PKC inhibitor composition may be in the form of tablets, pills, powders, liquid solutions or suspensions, suppositories, and injectable or infusible solutions. The preferred form depends on the intended mode of administration and therapeutic application.

### 3. Methods of Use

The invention discloses a method of treating prostate cancer in a subject. The method of treating prostate cancer in a subject may comprise administering the PKC inhibitor composition to the subject. The invention further discloses a method of preventing the development of prostate cancer in a subject. The method of preventing prostate cancer in a subject may comprise administering the PKC inhibitor composition to the subject.

The subject may be any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). The subject may be a human or a non-human. The subject or patient may be undergoing other forms of treatment.

The subject may be diagnosed with prostate cancer. The subject may be at risk of developing prostate cancer. For example, the subject may be diagnosed with a precancerous prostate state. The subject may be diagnosed with prostatic intraepithelial neoplasia (PIN). Prostatic intraepithelial neoplasia may be high grade PIN (HGPIN). The subject may be diagnosed with proliferative inflammatory atrophy (PIA). The subject may be diagnosed with atypical small acinar proliferation (ASAP).

The PKC inhibitor compositions may be administered to the subject by any suitable route that allows the PKC inhibitor to contact tumor cells. The compositions may be administered orally, parentally (including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, and epidural injection) by infusion, by electroporation, or co-administered as a component of any medical device or object to be inserted (temporarily or permanently) into a subject.

The PKC inhibitor composition may be administered independently or in combination with other anti-cancer therapies, including surgery, radiation therapy, chemotherapy, DNA therapy, adjuvant therapy, gene therapy, and additional therapeutic agents for the treatment of prostate cancer. Additional therapeutic agents may be alkylating agents, angiogenesis inhibitors, antibodies, antimetabolites, antimitotics, antiproliferatives, aurora kinase inhibitors, Bcl-2 family protein (for example, Bcl-xL, Bcl-2, Bcl-w) inhibitors, Bcr-Abl kinase inhibitors, biologic response modifiers, cyclin-dependent kinase inhibitors, cell cycle inhibitors, cyclooxygenase-2 inhibitors, leukemia viral oncogene homolog (ErbB2) receptor inhibitors, growth factor inhibitors, heat shock protein (HSP)-90 inhibitors, histone deacetylase (HDAC) inhibitors inhibitors, hormonal therapies, inhibitors of apoptosis proteins (IAPs), immunologicals, intercalating antibiotics, kinase inhibitors, mammalian target of rapamycin inhibitors, mitogen-activated extracellular signal-regulated kinase inhibitors, microRNA's, small inhibitory ribonucleic acids (siRNAs), non-steroidal anti-inflammatory drugs (NSAID's), poly ADP (adenosine diphosphate)-ribose polymerase (PARP) inhibitors, platinum chemotherapeutics, polo-like kinase inhibitors, proteasome inhibitors, purine analogs, pyrimidine analogs, receptor tyrosine kinase inhibitors, retinoids/deltoids plant alkaloids, topoisomerase inhibitors and the like.

The PKC inhibitor composition and other anti-cancer therapy may be administered simultaneously or sequentially to the subject. For example, the other anti-cancer therapy may be administered before the PKC inhibitor composition. The other anti-cancer therapy may be administered after the PKC inhibitor composition. Sequential administration may involve treatment with the other anti-cancer therapy on the same day (within 24 hours) of treatment with the PKC inhibitor composition. Sequential administration may also involve continued treatment with the other anti-cancer therapy on days that the PKC inhibitor composition is not administered.

Administration of the PKC inhibitor composition may be as a single dose, or multiple doses over a period of time. The PKC inhibitor composition may be administered to the patient at any frequency necessary to achieve the desired therapeutic effect. For example, the PKC inhibitor composition may be administered once to several times every month, every two weeks, every week, or every day. Administration of the PKC inhibitor composition may be repeated until the desired therapeutic effect has been achieved. For example, the PKC inhibitor composition may be administered once to several times over the course of 1 day, 3 days, 5 days, 1 week, 2 weeks, 3 weeks, 1 month, 2 months, 3 months, 4 months, 5 months, 6 months, 7 months, 8 months, 9 months, 10 months, 11 months, 12 months, or more.

The amount of the PKC inhibitor to be administered may depend on a variety of factors, such as the route of administration and the seriousness of the condition, and should be decided according to the judgment of the practitioner and each patient's circumstances. The PKC inhibitor composition may be administered in any amount suitable for the treatment of prostate cancer in a subject. Treatment may include any one or more of inhibiting neoplastic transformation of cells, slowing the growth and/or proliferation of cancer cells, reducing tumor size, reducing the number of cancer cells, inducing apoptosis in cancer cells, decreasing the level of one or more atypical protein kinases in the cancer cells, decreasing the level of PKC-τ or PKC-ζ in cancer cells, inhibiting or slowing the invasion of cancer cells into surrounding or neighboring tissues, inhibiting or slowing the metastatic spread of cancer cells into distant parts of the body, enhancing the therapeutic effect of chemotherapy medications, and prolonging cancer patient survival.

An effective amount of the PKC inhibitor composition may cause a partial improvement or a complete elimination of symptoms due to neoplasia/cancer. For example, an effective amount of PKC inhibitor composition may cause a 5%, 10%, 20%, 30%, 40%, 50%, 60%, 70%, 80%, 90%, 95%, 99% or 100% reduction in the rate of growth and/or proliferation of cancer cells. As another example, an effective amount of PKC inhibitor composition may cause a 5%, 10%, 20%, 30%, 40%, 50%, 60%, 70%, 80%, 90%, 95%, 99% or 100% reduction in tumor size.

Suitable dosage ranges of the PKC inhibitor composition include from about 0.001 mg PKC inhibitor/kg body weight to about 100 mg/kg, about 0.01 mg/kg to about 50 mg/kg, about 0.1 mg/kg to about 25 mg/kg, about 0.5 mg/kg to about 15 mg/kg, about 1 mg/kg to about 10 mg/kg, or about 2.5 mg/kg to about 5 mg/kg. In addition, in vitro assays may optionally be employed to help identify optimal dosage ranges. Effective doses may be extrapolated from dose-response curves derived from in vitro or animal model test systems.

### 4. Kits

The invention further discloses a kit, which may be used to treat prostate cancer or prevent the development of prostate cancer in a subject. The kit comprises at least the PKC inhibitor composition. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 5. Examples

The disclosed compounds, compositions and methods will be better understood by reference to the following examples, which are intended as an illustration of and not a limitation upon the scope of the invention. Where the term comprising is used herein, it should be understood that the disclosure also contemplates alternative embodiments consisting of or consisting essentially of the recited features.

### Example 1: Materials and Methods

### Materials:

ACPD or 2-acetyl-1,3-cyclopentanedione was purchased from Sigma-Aldrich (St. Louis, Mo.). It was dissolved in sterile distilled water before use. Dulbecco's phosphate buffered saline without Mg2+ and Ca2+(DPBS) was purchased from the American Type Culture Collection Rockville, Md.). Trypsin-EDTA (ethylenediaminetetraacetic acid) solution was purchased from Life Technologies (Carlsbad, Calif.). Polyclonal primary antibodies were purchased from the following companies: β-actin (sc-1616) goat polyclonal, PKC-α (sc-8393) mouse monoclonal (Santa Cruz Biotechnology, CA); Anti-PKC-τ mouse monoclonal catalog number 610176 (Transduction Laboratory, Lexington, Ky.). Secondary antibodies were purchased from the following companies: Horse radish Peroxidase (HRP) Goat×Mouse IgG catalog number JGM035146, HRP Goat×Rabbit IgG (catalog number JGZ035144) (Accurate, Westbury, N.Y.); HRP Bovine anti-goat IgG (sc-2350) (Santa Cruz Biotechnology, CA).

### Prostate Tissue Analysis:

Protein for Western Blotting was extracted from human biopsy derived benign prostate hyperplasia (BPH) tissues obtained from the Cooperative Human Tissue Network (Southern Division) at the University of Alabama at Birmingham. Tissue specimens were obtained from males of varying ages (57-80 years old). Protein extraction from fresh frozen radical prostatectomy specimens from patients with PC were obtained from patients operated at the James A. Haley Veterans Hospital in Tampa from May to August 2007, as part of a clinical protocol approved by the University of South Florida Institutional Review Board. The specimens were placed on ice immediately after prostectomy, frozen in liquid nitrogen within 30 minutes to 1 hour after prostectomy. Prostate tissues (0.5-1 g) were re-suspended and sonicated in 2 mL homogenization buffer (50 mM HEPES [pH 7.5], 150 mM NaCl, 0.1% Tween-20, 1 mM EDTA (ethylenediamine-tetraacetic acid) and 2 mM EGTA (ethylene glycol bis(β-aminoethyl ether)-N,N,N′,N′,-tetraacetic acid), 0.1 mM orthovanadate, 1 mM NaF, 2 mM PMSF(phenyl-methylsulfonly fluoride), 2.5 μg/ml leupeptin, 1 mM DTT (dithiothreitol), 0.15 U/mL aprotinin. The suspension was sonicated for 3 fifteen seconds cycles on ice. Brain tissue suspensions or cell lysates were centrifuged at 40,000 g for 30 min to obtain cell extracts. Protein content was measured according by the Bradford method. Tissue extracts containing equal amounts of protein in each lane were run on SDS-PAGE gels. The tissue lysates (100 μg) were Western blotted for PKC-α (10 μg, 1:500 dilution): and PKC-τ (5 μg, 1:4000 dilution) and β-actin (10 μg, 1:500 dilution). Secondary antibodies were obtained from Accurate (JGM035146, Westboro, N.Y. and JG2035744) and used at 1:15000 dilutions (1 μg).

### Immunohistochemical Staining:

For immunohistochemistry (IHC), de-identified, archival, unstained sections of full core prostate needle biopsies (PNB) were obtained for diagnostic purpose as part of a systematic PC screening program at the James A. Haley Veterans Administration Medical Center. The PNB specimens studied were from 10 patients with PC (1 core with adenocarcinoma from each patient), 8 patients with High Grade Prostate Intraepithelial Neoplasma (HGPIN) (1 core with HGPIN from each patient) and 9 patients with BPH (1 core with from each patient). Following deparaffinization of the sections, and citrate microwave antigen retrieval, blocking was performed. For detection of PKC-α, sections were incubated with anti-PKC-α (1:100 dilution) for 60 minutes followed by washing and detection with EnVision detection system using mouse IgG polymer and DAB chromogen. For detection of PKC-τ, separate sections were subsequently incubated over night with purified mouse anti-PKC-τ (1:200 dilution; BD transduction laboratories), followed by washing and subsequent 30 minute incubation with 1:200 rat anti-mouse IgG2b. Final detection was done using DAB chromogen. All sections were examined for PKC-α and PKC-τ expression and scored using Allred semi quantitative scoring system. The Allred score is a composite of the percentage of cells that stained and the intensity of their staining. The percentage of cells staining is called proportion score and is classified from 0 through 5, and the intensity of cells staining is called intensity score and is rated as 1, 2 or 3. The composite score ranges between 0-8 with 0 being the lowest and 8 being the maximum score. The adenocarcinoma glands, glands with HGPIN, benign glands and stromal cells were scored separately.

### Cell Culture:

RWPE epithelial cells and DU-145 human prostate carcinoma cells were purchased from American Type Culture Collection (ATCC). PC-3 cells were acquired from Moffit Cancer center. The cells were grown as a monolayer in a T25 tissue culture flask with 5 mL of growth medium and maintained in a 37 C incubator with 5% CO2. The E-MEM and F-12 growth media were obtained from ATCC. The medium was supplemented with 10% fetal bovine serum (FBS) and a mix of the antibiotics Penicillin (10,000 IU) and Streptomycin (10,000 μg/ml) in a 100× concentration which was purchased from Corning.

### Cell Proliferation Assay:

RWPE, DU 145 and PC-3 cells were cultured in a T25 cell culture flasks. 20,000 cells were seeded into each. Flasks were treated with either ICA-1 or ACPD in doses of 1 uM, 5 uM, and 10 uM along with a control set for each that were untreated. The treatment was repeated over the course of 72 hours and samples were taken at 24 hour intervals.

### Cell Counting:

RWPE, DU 145, and PC-3 cells were cultured in a T25 cell culture flasks. At assigned intervals the cells were trypsonized and put into a 1.5 mL microcentrifuge tube. Cells were inverted gently to keep the solution homogeneous and then the Cell Scepter by Millipore was used to measure cell numbers.

### Western Blot Analysis:

Protein was harvested from cultured cells using Lysis Buffer purchased from Cell Signaling. Protein content was measured according to the Bradford method. Tissue extracts containing equal amounts of protein in each lane were run on SDS-PAGE gels. The tissue lysates (50 μg) were Western blotted for PKC-τ (10 μg, 1:2500 dilution): and PKC-ζ (5 μg, 1:4000 dilution) and β-actin (10 μg, 1:3000 dilution).

### Cell Subfractionation:

DU 145 and PC-3 cells were cultured in a T25 cell culture flasks. 20,000 cells were seeded into each. Flasks were treated with either ICA-1 or ACPD at a dose 10 μM along with a control set for each that was untreated. The treatment was repeated over the course of 71 hours and samples were taken at 24 hour intervals. Thirty minutes prior to harvesting the cells were exposed to 10 nM of TNF-α. The NE-PER Nuclear and Cytoplasmic Extraction Kit from thermo scientific was used to harvest the protein and separate the nuclear from the cytoplasmic proteins.

### Densitometry:

Western blots were developed on the Protein Simple Flour Chem M. Accompanied by the Alpha view software for analysis.

### Statistical Analysis:

Homoscedasticity was determined by F-test. Statistical significance was determined by T-test.

### Example 2: PKC-τ Expression in Prostate Tissue

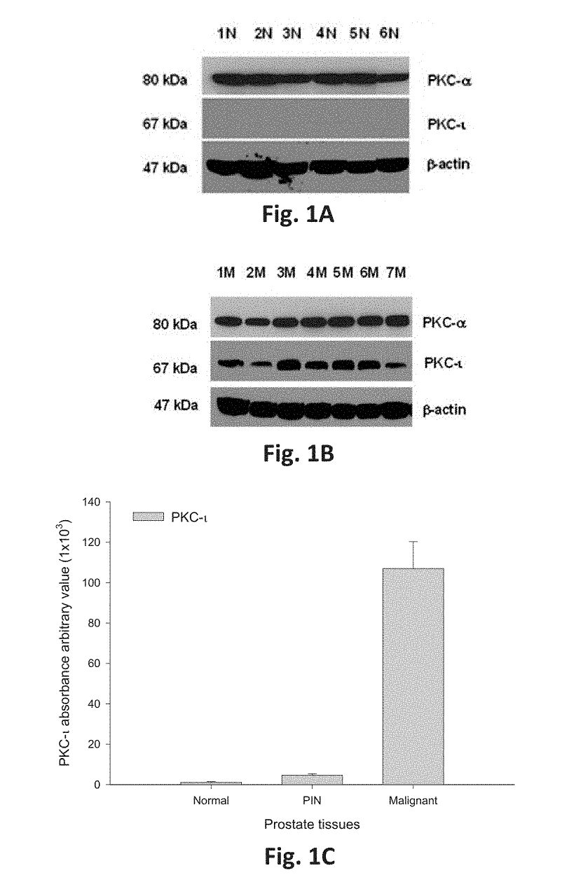

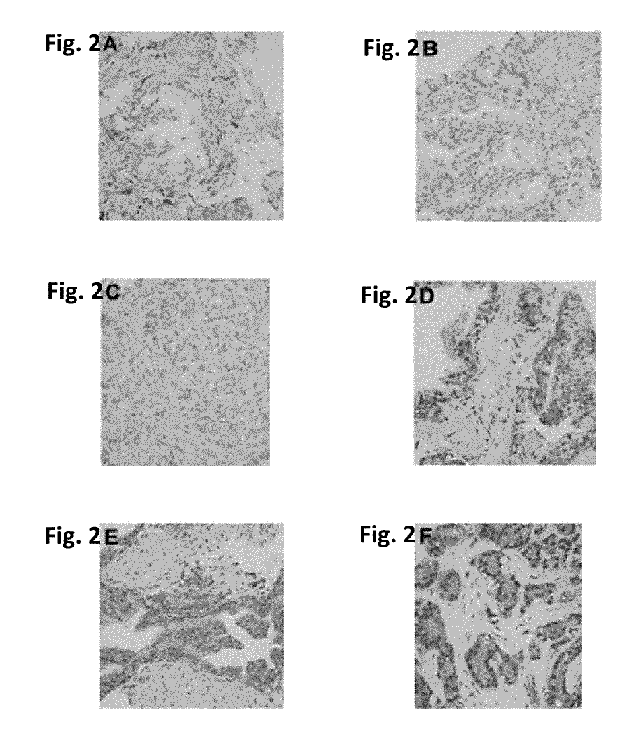

Western Blot: Expression of the atypical protein kinase PKC-iota was assessed in benign prostate hyperplasia (BPH, n=6) tissue and malignant prostate cancer tissue (PC, n=7). Protein was extracted from tissue and the tissue lysates were Western blotted for PKC-α, PKC-τ, and β-actin. Results are shown in FIGS. 1A-C. PKC-α (positive control PKC) and PKC-τ were identified in Western blots by bands with molecular weights of 80 kD and 67 kD, respectively. PKC-τ shows little expression in BPH tissue (“N”, FIG. 1A) and abundant expression in all malignant prostate tissue (“M”, FIG. 1B). PKC-α did not show significant changes in abundance in malignant prostate tissue compared to BPH controls. Control β-actin Western blots showed β-actin immunoreactive bands at a molecular weight of 42 kD. The β-actin immunoreactive bands were of equal intensity, indicating that equal amount of protein were loaded into each lane. The data presented in FIGS. 1A-1B are quantified in FIG. 1C, which depicts a 100 fold increase in PKC-τ immunoreactivity in malignant prostate tissue when compared to BPH tissue. Data was analyzed by student t-test (n=6 BPH, 7PC, p=0.00048). Taken together, this data indicates that PKC-τ is overexpressed in malignant prostate tissue compared to BPH tissues.

### Immunohistochemistry:

Subsequently, IHC was performed to investigate comparatively the tissue distribution and intracellular localization of PKC-α and PKC-τ. Samples from patients with High Grade Prostate Intraepithelial Neoplasma (HGPIN, n=8), BPH (n=9), and PC (n=10) were stained for PKC-iota and PKC-α as a control. PKC-α staining in BPH, HGPIN, and PC tissue is shown in FIGS. 2A-C, respectively. PKC-τ staining in BPH, HGPIN, and PC tissue is shown in FIGS. 2D-F, respectively. Details are further highlighted in table 2.

The IHC data illustrate that PKC-α was expressed in the stromal cells but minimal expression was noted in majority of BPH, PC, and HGPIN glands. In only one case did the PC glands show moderate expression of PKC-α. These PC glands expressing PKC-α had a Gleason pattern 4 (Table 2). The increase in PKC-α expression in this case may be due to gene amplification or genetic duplication of PKC-α.

In contrast, PKC-τ expression was noted in all glands in PC tissue (proportion score of 5 in the majority of cases; Table 1 and FIG. 2F). The intensity of staining was stronger in the PC glands with Allred scores of +8 and +7 (Table 2 and FIG. 2F) compared to benign glands (FIG. 2D) and glands with HGPIN (FIG. 2E), as further shown in Table 2. The observation of very strong and consistent PKC-τ expression in all PC and some HGPIN specimens is significant, as it may allow prediction of the portion of HGPIN patients who will progress to clinical PC. This may offer an opportunity to target it in HGPIN patients for the prevention of PC.

Overall, the IHC data confirm the higher expression of PKC-τ observed in PC using Western blotting from fresh frozen excisional clinical prostate tissue specimens.

TABLE 1
Status of PKC-ι in BPH, PIN and malignant prostate tissues*

| | Not | | |
|---|---|---|---|
| Tissue Type | Present | Weakly Present | Positively Present |
| BHP | 6 | ∘ | 0 |
| PIN | 1 | 0 | 2 |
| Malignant | 0 | 0 | 7 |
| Prostate | | | |

*Prostate tissue was obtained from prostate chips or prostatectomy.

TABLE 2
PKC-α and PKC-t staining of glands and stroma of patients
with BPH, HGPIN and prostate adenocarcinoma

| | Total | | | | | | | | | |
|---|---|---|---|---|---|---|---|---|---|---|
| | cases | 8+ | 7+ | 6+ | 5+ | 4+ | 3+ | 2+ | 1+ | 0 |
| | | PKC-α staining (no. of cases)* | | | | | | | | |
| Benign | 9 | | | | | | | | | |
| Glands | | 0 | 0 | 0 | 0 | 0 | 0 | 5 | 0 | 4 |
| Stroma | | 0 | 1 | 2 | 5 | 1 | 0 | 0 | 0 | 0 |
| HGPIN | 8 | | | | | | | | | |
| HGPIN glands | | 0 | 0 | 0 | 0 | 0 | 0 | 3 | 0 | 5 |
| Stroma | | 0 | 1 | 3 | 4 | 0 | 0 | 0 | 0 | 0 |
| Adenocarcinoma | 10 | | | | | | | | | |
| Tumor glands | | 0 | 0 | 1 | 0 | 1 | 0 | 4 | 0 | 4 |
| Stroma | | 0 | 1 | 4 | 2 | 1 | 0 | 2 | 0 | 0 |
| | | PKC-t staining (no. of cases)* | | | | | | | | |
| Benign | 9 | | | | | | | | | |
| Glands | | 0 | 2 | 7 | 0 | 0 | 0 | 0 | 0 | 0 |
| Stroma | | 0 | 0 | 0 | 2 | 4 | 2 | 1 | 0 | 0 |
| HGPIN | 8 | | | | | | | | | |
| HGPIN glands | | 1 | 3 | 3 | 1 | 0 | 0 | 0 | 0 | 0 |
| Stroma | | 0 | 0 | 0 | 0 | 3 | 4 | 1 | 0 | 0 |
| Adenocarcinoma | 10 | | | | | | | | | |
| Tumor glands | | 9 | 1 | 0 | 0 | 0 | 0 | 0 | 0 | 0 |
| Stroma | | 0 | 0 | 0 | 3 | 5 | 2 | 0 | 0 | 0 |

*Staining based on the Allred score [38].

### Example 3: Inhibition of aPKCs in Prostate Cells

### Measurement of PKC-iota:

The ability of ICA-1 and ACPD to effect levels of PKC-iota was assessed in prostate cells. DU-145 prostate carcinoma cells and non-malignant prostate RWPE-1 cells were cultivated in separate flasks and treated with ICA-1 or ACPD for three consecutive days. ICA-1 was used as a PKC-iota inhibitor. ACPD was used as a pan-aPKC inhibitor that inhibits both PKC-iota and PKC-zeta. The cells were counted before and after the treatments and a statistical analysis was performed. Following cell counting, the cells were lysed and levels of PKC-iota and PKC-zeta were measured by Western blotting and immunoprecipitation.

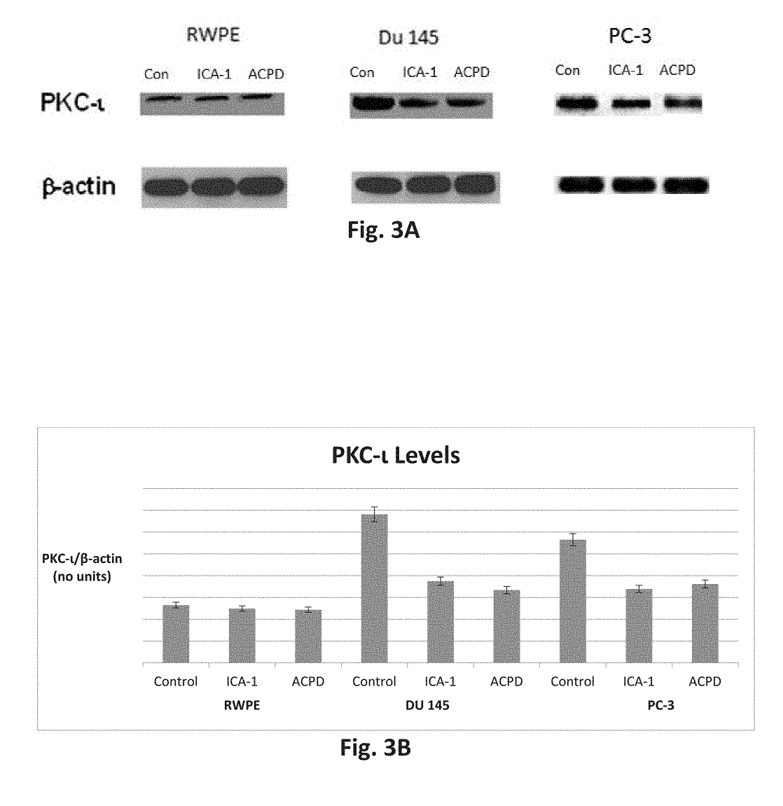

Assessment of PKC-iota levels is depicted in FIG. 3. Western blots are shown in FIG. 3A, and results are quantified in FIG. 3B. In the DU-145 and PC-3 cells, there is an abundance of PKC-τ compared to RWPE cells. Levels of PKC t were assessed 72 hours after treatment with ICA-1 or ACPD. RWPE cells display a slight reduction in PKC-iota following treatment but neither drug reduced the levels of PKC-τ by more than 10%. In contrast, DU-145 cells show an average of 46% decrease in PKC t following treatment with ICA-1, and an average of 51% decrease following treatment with ACPD. In PC-3 cells there is a 40% decrease in PKC t with treatment of ICA-1 and a 37% decrease of PKC I with treatment of ACPD. There was no significant difference in β-actin between the treated and untreated samples, indicating that equal amounts of protein were loaded into each well.

### Dose-Response Curves:

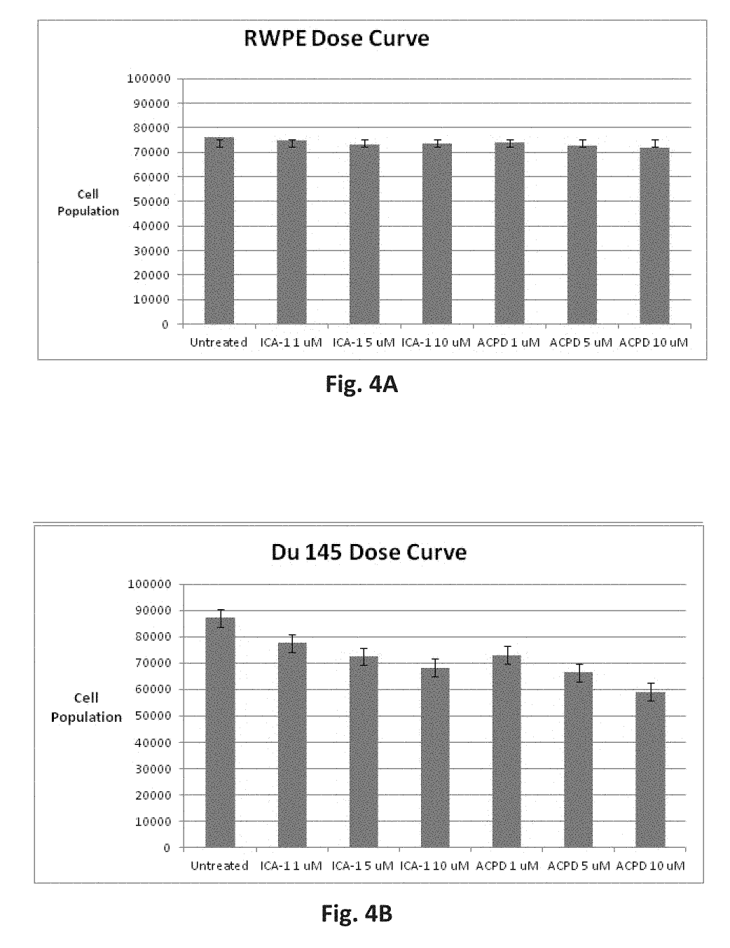

Dose-response curves were generated to measure the concentration of drugs needed for an effective response in malignant cancer cells while having no significant effect on control cells. FIG. 4A shows the dose-response curve for ICA-1 and ACPD treatment in RWPE cells. FIG. 4B shows the dose-response curve for ICA-1 and ACPD treatment in DU-145 cells. For both cell lines, an increase in dosage of ICA-1 and ACPD led to a corresponding increase in cell death. At 10 μM both drugs showed a statistically significant decrease in population in malignant cells while not having a significant decrease in non-malignant cells.

### Cell Growth:

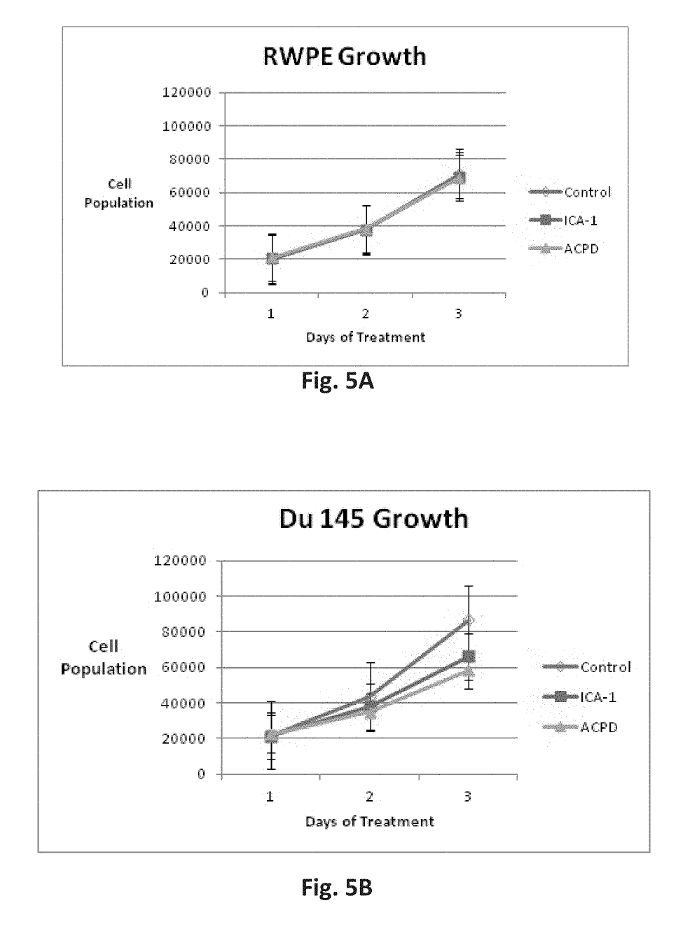

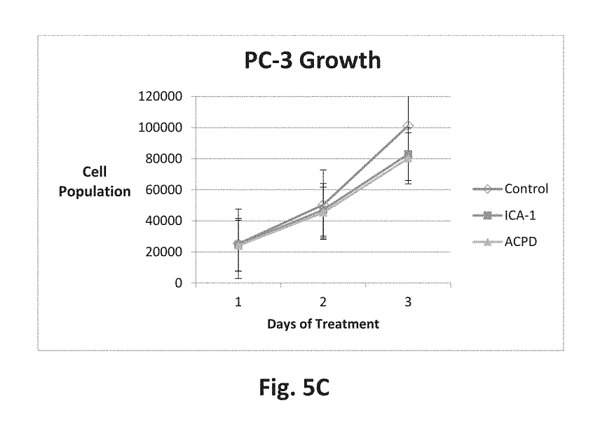

The average growth of RWPE, DU-145, and PC-3 cell lines under control conditions, treatment with ICA-1, and treatment with ACPD is shown in FIGS. 5A-C, respectively. RWPE cells treated with ICA-1 and ACPD display a minor but noticeable reduction in cell population growth compared to untreated cells. Both inhibitors retain over 90% of the cell population present in the untreated RWPE cells. In contrast, there is a substantial decrease in cell population of DU-145 cells treated with the inhibitors. DU-145 cells treated with ICA-1 display a 19% reduction in cell growth, while cells treated with ACDP display a 32% decrease in cell growth compared to untreated cells. PC-3 cells show a similar trend of population decline, although there was not as large a difference between drugs. Treatment with ICA-1 showed an average decline of 18%. Treatment with ACPD showed an average decline of 21%.

### Apoptotic Markers:

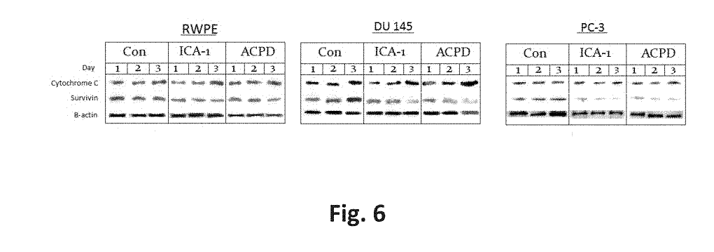

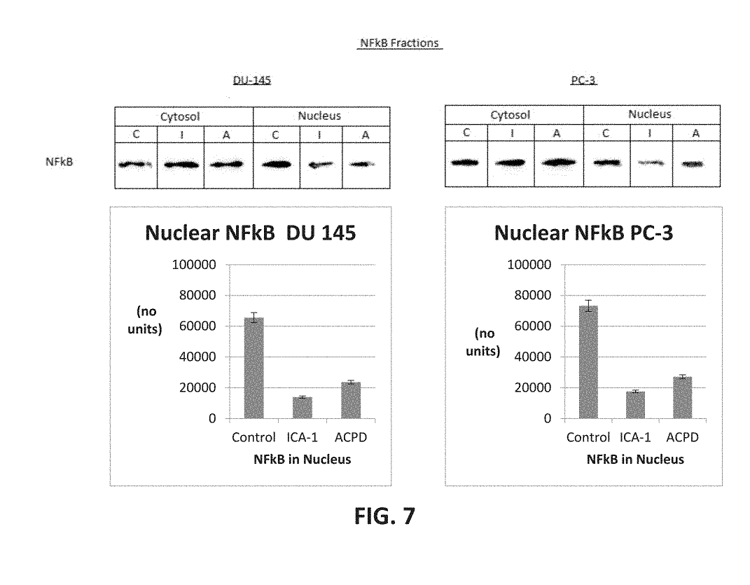

PKC-iota may be an anti-apoptotic factor that enables cancer cells to evade apoptosis. As such, RWPE control cells and DU-145 cancer cells were probed for apoptotic markers Cytochrome C and Survivin over the course of 3 days (72 hours) of treatment with ICA-1 and ACPD. Results are shown in FIG. 6. In all three cell lines treated with ICA-1 and ACPD there was a measurable increase in apoptosis. After 72 hours of treatment, the control line RWPE showed an 8% increase of Cytochrome C and a corresponding 10% decrease in Survivin with treatment of ICA-1. After 72 hours of treatment with ACPD, RWPE cells showed a 5% increase of Cytochrome C and a 7% decrease in Survivin. In contrast the cell line Du-145 showed a more substantial change with an almost 3-fold increase in Cytochrome C and a reduction of Survivin by half with treatment of ICA-1. After 72 hours of treatment with ACPD the Du-145 cells showed an average 400% increase in Cytochrome C and a 50% decrease in Survivin. In the PC-3 cells treated with ICA-1 there was a 2-fold increase in Cytochrome C and a 40% decrease in Survivin. PC-3 cells treated with ACPD showed a 150% increase in Cytochrome C and a 35% decrease in Survivin. All cell lines were also probed with β-actin to show equal loading between lanes.

### NF-kB Translocation:

The NF-kB signaling pathway is thought to play a role in development of different types of cancers, but its role in prostate cancer development is unknown. TNF-induced NF-κB activation is initiated by activation of inhibitor of κB (IκB) kinase (IKK). Activation of IKK causes the NF-κB nuclear localization signal to be exposed, allowing nuclear translocation of NF-kB and subsequent transcription of its target genes. Several target genes of NF-kB, including A20, cIAP-1, cIAP-2, Bcl-xL, XIAP, and IEX-1L, are known to have anti-apoptotic properties which allow cancerous cells to evade apoptosis.

To assess the impact of aPKC inhibition of NF-kB translocation, DU-145 and PC-3 cell lines were treated with ICA-1 or ACPD and exposed to TNF-α to activate NF-kB. Cytosolic and nucleic proteins were fractionated and levels of NF-kB were assessed. After exposure to TNF-α both DU 145 and PC-3 cells shows an increase in NF-kB in the nucleus and a decrease in the cytosol (C). DU-145 and PC-3 cells treated with ICA-1 (I) or ACPD (A) showed a decrease in translocation of NF-kB to the nucleus compared to untreated control cells. Treatment with ICA-1 there caused a 5-fold decrease of nucleic NFkB, whereas ACPD caused a 3-fold decrease of nucleic NFkB. This data suggests that by inhibiting PKC-τ, the transcription factor NF-kB remains inhibited. This inhibition could explain the decrease in cell populations for both cell lines treated with PKC-τ inhibitors.

For reasons of completeness, various aspects of the invention are set out in the following numbered clauses:

Clause 1: A method of treating prostate cancer in a subject, the method comprising administering to the subject a composition comprising a protein kinase C (PKC) inhibitor.

Clause 2: The method of clause 1, wherein the PKC inhibitor inhibits at least one atypical PKC (aPKC).

Clause 3: The method of clause 2, wherein the PKC inhibitor inhibits PKC-iota.

Clause 4: The method of clause 3, wherein the PKC inhibitor is ICA-1 or a salt thereof.

Clause 5: The method of clause 1, wherein the PKC inhibitor is a pan-aPKC inhibitor.

Clause 6: The method of clause 5, wherein the pan-aPKC inhibitor inhibits at least PKC-iota and PKC-zeta.

Clause 7: The method of clause 5, wherein the pan-aPKC inhibitor is 2-acetyl-1,3-cyclopentanedione (ACPD) or a salt thereof.

Clause 8: The method of clause 1, wherein the subject is a mammal.

Clause 9: The method of clause 8, wherein the subject is a human.

Clause 10: The method of clause 1, wherein the PKC inhibitor is administered in combination with at least one other anti-cancer therapy.

Clause 11: A method of preventing the development of prostate cancer in a subject, the method comprising administering to the subject a composition comprising a PKC inhibitor.

Clause 12: The method of clause 11, wherein the subject is diagnosed with a precancerous prostate state.

Clause 13: The method of clause 12, wherein the precancerous prostate state is prostatic intraepithelial neoplasia (PIN).

Clause 14: The method of clause 11, wherein the PKC inhibitor inhibits at least one atypical protein kinase (aPKC).

Clause 15: The method of clause 14, wherein the PKC inhibitor inhibits PKC-iota.

Clause 16: The method of clause 15, wherein the PKC inhibitor is ICA-1 or a salt thereof.

Clause 17: The method of clause 11, wherein the PKC inhibitor is a pan-aPKC inhibitor.

Clause 18: The method of clause 17, wherein the pan-aPKC inhibitor inhibits at least PKC-iota and PKC-zeta.

Clause 19: The method of clause 17, wherein the pan-aPKC inhibitor is 2-acetyl-1,3-cyclopentanedione (ACPD) or a salt thereof.

Clause 20: The method of clause 11, wherein the subject is a mammal.

Clause 21: The method of clause 20, wherein the subject is a human.

What is claimed is:

1. A method of treating prostate cancer in a subject, the method comprising administering to the subject a composition comprising a protein kinase C (PKC) inhibitor.

2. The method of claim 1, wherein the PKC inhibitor inhibits at least one atypical PKC (aPKC).

3. The method of claim 2, wherein the PKC inhibitor inhibits PKC-iota.

4. The method of claim 3, wherein the PKC inhibitor is ICA-1 or a salt thereof.

5. The method of claim 1, wherein the PKC inhibitor is a pan-aPKC inhibitor.

6. The method of claim 5, wherein the pan-aPKC inhibitor inhibits at least PKC-iota and PKC-zeta.

7. The method of claim 5, wherein the pan-aPKC inhibitor is 2-acetyl-1,3-cyclopentanedione (ACPD) or a salt thereof.

8. The method of claim 1, wherein the subject is a mammal.

9. The method of claim 8, wherein the subject is a human.

10. The method of claim 1, wherein the PKC inhibitor is administered in combination with at least one other anti-cancer therapy.

11. A method of preventing the development of prostate cancer in a subject, the method comprising administering to the subject a composition comprising a PKC inhibitor.

12. The method of claim 11, wherein the subject is diagnosed with a precancerous prostate state.

13. The method of claim 12, wherein the precancerous prostate state is prostatic intraepithelial neoplasia (PIN).

14. The method of claim 11, wherein the PKC inhibitor inhibits at least one atypical protein kinase (aPKC).

15. The method of claim 14, wherein the PKC inhibitor inhibits PKC-iota.

16. The method of claim 15, wherein the PKC inhibitor is ICA-1 or a salt thereof.

17. The method of claim 11, wherein the PKC inhibitor is a pan-aPKC inhibitor.

18. The method of claim 17, wherein the pan-aPKC inhibitor inhibits at least PKC-iota and PKC-zeta.

19. The method of claim 17, wherein the pan-aPKC inhibitor is 2-acetyl-1,3-cyclopentanedione (ACPD) or a salt thereof.

20. The method of claim 11, wherein the subject is a mammal.

21. The method of claim 20, wherein the subject is a human.
